(19) United States
(12) Patent Application Publication
(10) Pub. No.: US 2011/0196188 A1
(43) Pub. Date: Aug. 11, 2011

(54) METHODS AND APPARATUS TO CREATE RESONANCE IN WATER AND TO DESTROY RESONANCE IN BACTERIA

(76) Inventor: Hans Giertz, Bromma (SE)

(21) Appl. No.: 13/123,024

(22) PCT Filed: Oct. 5, 2009
(86) PCT No.: PCT/SE2009/000439

§ 371 Date: Apr. 7, 2011

(30) Foreign Application Priority Data
Oct. 8, 2008 (SE) 0802118-0
May 19, 2009 (SE) 0900675-0

(51) Int. Cl. A61N 2/02; A61L 2/08

(52) U.S. Cl. 600/13; 422/22

(57) ABSTRACT

A method to create resonance in water at the period 24/3−2¹⁷ hour, wherein said water is exposed, during a limited time, to electromagnetic energy having the period 24/3−2¹⁷ hour, resulting in that continued resonance in said water is maintained by electromagnetic energy in the air having the period 24/3−2¹⁷ hour and where said water include sea water, lake water, river water, water in wells, water in reservoirs, water in beverages, water in food, water in human body fluids and cells, water in other types of species and organisms and molecule structures consisting of H₂O molecules and ions.

### TECHNICAL FIELD

The present invention relates to the fields of resonance in water, and more specific to a method to create resonance in water according to the preamble of claim 1.

### BACKGROUND AND PRIOR ART

It's known that the rotation of the earth creates currents with the period 24 hours in the magnetosphere, known as the Birkeland current. Plasma winds in the ionosphere create currents of millions of ampere having the period 24 hours, called Sq current, and it can be measured as diurnal variations in the geomagnetic field.

The global electric circuit is known since the 1920ies and it supplies the earth crust and the air with charge; electrons, negative and positive ions. The 200 kV potential between the atmosphere and earth creates a vertical current density approx. 3 pA/m².

It's known that H₂O forms molecule structures (ex. tetrahedral structure), that water is polar in nature and dissolves salts (ex. NaCl) into ions where Na⁺ is attracted to ^{δ-}O and Cl⁻ to ^{δ+}H and H₂O molecules are bonded by the extremely weak hydrogen bond.

It's known that some biological processes such as the water channel, ion channels, receptors and the blood-brain-barrier depend on specific H₂O/ion molecule structures and that these processes are influenced by low frequency electromagnetic energy.

There exist no methods which allow measurement of extremely low amplitude and very low frequency electromagnetic displacement and conduction current (≦10 Hz). Therefore the above phenomena and their mutual relationship are poorly understood.

### SUMMARY OF THE INVENTION

### Enabler

The inventor has developed methods which enable measurement and description of processes based on extremely small amplitude conduction and displacement current having very low frequency. Many electromagnetic biological processes operate at extremely low amplitude and low frequency and the invention is based on the understanding of electromagnetic processes that have not been described or reported before.

### SUMMARY OF THE INVENTION

### Object of the Invention

The object of this invention is to create a method which creates resonance in water and where the resonance is maintained by electromagnetic energy in the air at the period 24/3·2¹⁷ hour=4.55 Hz.

This is achieved with the method according to the invention as defined in claim 1.

Resonance at the period 24/3·2¹⁷ hour relates to specific molecule structures. Thus ions must be released from existing bonds to H₂O molecules and transferred to new bonds and new molecule structures. According to one embodiment of the invention, as defined in claim 2 this process is improved when the water is exposed to electromagnetic energy in the period 0.5-0.1 s (2-10 Hz) range and preferably the period 24/2¹⁸ hour=3.03 Hz. According to a further embodiment of the invention, as defined in claim 7, said water is exposed to mechanical waves, preferably sound waves having a frequency f, 0.1 kHz≦f≦5 kHz, which also improves the above process.

The invention encompass all water including sea water, lake water, river water, water in wells, water in reservoirs, water in beverages, water in food, water in human body fluids and cells, water in other species and organisms and in molecule structures consisting of a few or many H₂O molecules and ions.

The object of this invention is also to create methods which limits and/or destroys the ability of organisms to create resonance with electromagnetic energy in the air. The method is to expose said organisms to electromagnetic energy having the period 24/3·2¹⁷ hour and the period 24/2¹⁸ hour as defined in claim 3, alternatively to electromagnetic energy in the period 0.5-0.1 s range as defined in claim 9. Said organisms include all types of types of disease-causing pathogens causing infections and inflammations, all types of bacteria, all types of viruses, all types of cancer cells and tumors, all types of mould and all types of fungal microbes.

### SUMMARY OF THE INVENTION

### Short Description

The invention builds on the observation that the air contains extremely low amplitude and low frequency electromagnetic energy consisting of conduction current having periods equal to harmonics of the period 24 hours, i.e. the periods 24/m·2ⁿ hours. Water can be described as a forced damped oscillator and in order to create resonance electromagnetic energy must be injected and the energy needed to maintain resonance is extremely small. Once water is in resonance, the resonance is maintained by electromagnetic energy in the air. However, in order to create resonance ions must be moved into new positions (the molecule structure is changed) and the needed amplitude of the electromagnetic energy is significantly higher than the energy present in the air. This is explained in detail in the chapter; Detailed Theory.

Resonance in water can be obtained in the period 0.5-0.1 s range (i.e. 2-10 Hz range) by exposing water to electromagnetic energy having sufficient amplitude at said period. The electromagnetic energy can be electric fields, magnetic fields, conduction current or combinations of these energy forms. When the injected energy has the same period as one of the harmonics of the energy in the air (i.e. the period 24/m·2ⁿ hours and within the period 0.5-0.1 s range) conduction current in the air propagates to the water in resonance (because its impedance is low at resonance) and this conduction current maintains the resonance when the injection of the energy ceases. The air contains electromagnetic energy caused by the rotation of the earth with the period 24 hours having large amount of harmonics and the following harmonics create and maintain resonance in water; the periods 24/2¹⁸ hour=3.03 Hz, 24/3·2¹⁷ hour=4.55 Hz, 24/5·2¹⁶ hour=3.79 Hz, 24/7·2¹⁶ hour=5.31 Hz and 24/9·2¹⁵ hour=3.41 Hz.

Humans and many other species have a build in mechanism (oscillator) that creates resonance at the period 24/3·2¹⁷ hour=4.55 Hz. This creates resonance with the electromagnetic energy in the air and conduction current with relatively high amplitude connects to the oscillator. This creates resonance in water in the vicinity of the oscillator, i.e. in the brain and this water is distributed by the blood into the entire body. Therefore the water in humans and said species normally has resonance at said period and this resonance is maintained by energy in the air. A number of processes depend on this resonance in water and the water molecule structures that it creates. Some processes depend on the resonance period of water consumed in food and beverages. Other species contain biological processes that depend on the resonance in water in their surroundings. A number of factors influence the resonance in water in humans, in other species, in water consumed and in the environment and said resonance is often disturbed or the resonance ceases completely. Creating resonance in said water at the period 24/3·2¹⁷ hour improves said processes and that is explained in the chapter; Detailed Theory.

Resonance can be created in almost all types of water including sea water, lake water, river water, water in wells, water in reservoirs, water in beverages, water in food, water in human body fluids and cells, water in other species and organisms and in molecule structures consisting of a few or many H₂O molecules and ions.

Organisms like bacteria, viruses, cancer cells, mould and fungal microbes have a build in oscillator that creates resonance at a period deviating from the period 24/3·2¹⁷ hour and therefore said organisms change the resonance period of the water in their vicinity and this disturbs biological processes in the host. Exposing said organisms to electromagnetic energy with specific period and duration always leads to the same result; said organisms lose their ability to create resonance and the ability to create resonance never regains. The procedure can be performed in vivo and in vitro. At this stage the exact reason is unknown; does the procedure destroy the oscillator or important biological processes or does it kill said organism? The end result is that the immune system is strengthened and the illness is rapidly cured. The process that destroys the ability to create resonance is (with large probability) that the water and oscillator in said organisms is forced to oscillate at a period deviating from the natural resonance period of said organisms and it influences biological processes. This is explained in Detailed Theory.

### SUMMARY OF THE INVENTION

### Achieved Results

The human body can be regarded as an electromagnetic resonance system and the amplitude of the resonance at the period 24/3·2¹⁷ hour is an indication of the volume of water in resonance and the Q-value of the water, i.e. how well the water molecule structures are adapted to the resonance as well as disturbance from other electromagnetic resonance systems in the body. Small variations in resonance amplitude have little influence but large decrease is an indication of disturbance which can impact biological processes. The following has been observed.

A meal with food and beverages that contain water not having resonance normally decreases the amplitude of resonance at the period 24/3·2¹⁷ hour in a person's water with ≈10-25%. A meal containing water in resonance at the period 24/3·2¹⁷ hour increase the amplitude of resonance in a person's water with ≈10%. Consumed mineral water has larger impact because of its high ion content. The figures are indicative and depend on the person's weight, amount of water consumed in the meal etc. How this is experienced is up to the individual.

Food and beverages that contain water having resonance at the period 24/3·2¹⁷ hour sometimes taste better, more natural and more fresh. The experience depends on the individual and also on the quality of the food and beverage. In general poor quality food and beverages do not improve, good quality improve in taste.

Disease-causing organisms, i.e. pathogens such as pathogenic bacteria, pathogenic viruses, fungal microbes and cancer change the resonance period in water and the water molecule structures in the vicinity of the wound and this disturbs biological processes in the host. Electromagnetic energy having the period 24/3·2¹⁷ hour, injected into the wound, create resonance in the water (in body fluids and cells) at said period and it improves the function of some biological processes in the host. The electromagnetic energy is injected a number of times (ex. during 30 seconds, 3 times daily in 10 days). Normally it strengthens the person's the immune system sufficiently to cope with the illness or the pathogens.

The vast majority of bacteria are harmless or beneficial. A large number of experiments have been performed on a variety of harmless and pathogen bacteria in vitro. A cluster of bacteria is measured with respect to resonance period and amplitude. Then the bacteria are exposed, during 24-96 hours, to electromagnetic energy in the period 0.5-0.1 s range and with a period deviating from the resonance period of the bacteria. The bacteria always lose their ability to create resonance. In most cases the bacteria have been observed during 1-4 weeks and they never regain their ability to create resonance. No exception has been observed.

Different types of mould have been treated in the same way with similar result.

Disease-causing organisms, i.e. pathogens in infections and inflammations, pathogenic bacteria, pathogenic viruses and cancer have been measured in vivo. Pathogens in humans, according to measurements made by the inventor, always create resonance at one of the following periods; 24/5·2¹⁶ hour=3.79 Hz, 24/7·2¹⁶ hour=5.31 Hz or 24/9·2¹⁵ hour=3.41 Hz but never at the period 24/2¹⁸ hour=3.03 Hz and the period 24/3·2¹⁷ hour=4.55 Hz. Thus said pathogens always create resonance at a period different to the resonance in water in the host (human). Therefore these pathogens can be exposed to electromagnetic energy with the period 24/3·2¹⁷ hour (and the period 24/2¹⁸ hour). The period 24/3·2¹⁷ hour is beneficial to the host (human) because it has no negative influence on the host's biological processes and no negative impact on the large number of harmless and beneficial bacteria (having resonance at the period 24/3·2¹⁷ hour) which live in symbioses with the host. The procedure used is to expose the infected area to electromagnetic energy with period 24/3·2¹⁷ hour during 30 seconds and the procedure is repeated 3 times daily in 10 days. The progress can be observed by measuring the resonance amplitude of the infected area and how said amplitude gradually decreases and normally the resonance created by said pathogens ceases completely after approx. 5 days. The illness is healed completely after 10-20 days. The procedure has been used on bacteria, viruses (ex. herpes) and cancer involving a large number of persons. In all cases the resonance created by the pathogens and cancer ceases completely after a few days and in all cases, except one, the persons consider themselves cured. In some cases the person would cure anyway but in many cases chronic inflammations, carried for months or years have been cured, herpes carried for 5 years has been cured as well as one case of cancer (malignant tumor).

It's important to distinguish between different mechanisms involved. This invention builds on the observation that organisms including pathogens contain oscillators that create resonance with electromagnetic energy in the air having the period 24/m·2ⁿ hour and that the ability to create resonance is disturbed when said organisms are exposed to electromagnetic energy having a period close to the resonance period of said organisms and permanently destroyed at prolonged exposure.

This is easily verified by electromagnetic measurements and the process can be monitored continuously in a precise way. The electromagnetic resonance from said organisms is measured in vitro or in vivo. In the latter case the electromagnetic resonance created by the pathogens is measured outside the body, thus the process is monitored without disturbing the patient or the process within the pathogens. There are secondary mechanisms involved; it has been observed that when said pathogens cease to create resonance they also die and the immune system in the host is strengthened and it leads to rapid cure.

Creating resonance at the period 24/3·2¹⁷ hour in household water (tap water), in wells, water reservoirs etc. is a matter of the experienced taste but it also has positive impact on water quality because it impacts the growth of bacteria.

### DETAILED THEORY

### Introduction

The air contains low frequency and extremely low amplitude electromagnetic energy consisting of conduction current J_{Air} in the air. J_{Air} contains a large amount of harmonics, among others at the following periods/frequencies; 24/2¹⁸ hour=3.03 Hz, 24/3·2¹⁷ hour=4.55 Hz, 24/5·2¹⁶ hour=3.79 Hz, 24/7·2¹⁶ hour=5.31 Hz and 24/9·2¹⁵ hour=3.41 Hz and this energy influences a number of processes in the human body and in other species. Water creates resonance in the same frequency range as the energy in the air J_{Air} and that is explained in the first part; Water. Some cell types contain oscillators that create resonance with J_{Air} and thus the cell absorbs energy in J_{Air}. Cluster of cells and the absorbed energy create resonance in water in their vicinity and this influence a number of biological processes. This is explained in the second part; Cells. It's essential to understand the nature of J_{Air} and that is explained in the last part; Air.

The processes described in this invention originate from energy created by the rotation of the earth having period 24 hours and harmonics having the periods 24/m·2ⁿ hours, m=1, 3, 5, 7 and 9 and therefore period instead of frequency is often used.

### DETAILED THEORY

### Water

Water creates electromagnetic resonance in the 2-10 Hz range, i.e. the same frequency range as harmonics in J_{Air}. Water contains angular molecules (H₂O) in which hydrogen and oxygen is joined by single covalent bonds where electron pairs making up the covalent bond create a small residual or partial positive charge (δ+) on the hydrogen atom, and a partial negative charge (δ−) on the oxygen atom. Water can dissolve a large number of ionic substances because of the interaction between the appropriate polarized end of the water molecule and ions. For example sodium chloride (Na⁺Cl⁻) solve in water with Na⁺ attracted to ^{δ-}O and Cl⁻ to ^{δ+}H. The polarized ends of H₂O create an extremely small force that bonds H₂O molecules called the hydrogen bond and this bond is elastic. Thus water can be described as a dielectric media having charge, mass and elasticity and the classical model based on a damped oscillator can be used, as described by Melrose and McPhedran^{l}. Consider a classical oscillator that corresponds to a mass m with charge q at a displacement X(t) from its mean position. Let the frequency of the oscillator be ω₀, and let it be damped with the decay constant γ. The oscillator is assumed to be forced by an electric field E(t). The equation of motion is X″(t)+γX′(t)+ω²₀X(t)=qE(t)/m. When the decay constant γ is small the mass (and charge) oscillates at its natural frequency ω₀=√k/m where k is the elasticity (spring constant). It's the electromagnetic energy E(t) acting on the charge q that supplies the oscillator with energy and the oscillator resonance frequency ω₀ is determined by its mechanical properties m and k. The oscillator is in resonance with the applied energy when the energy E(t) has a frequency ω=ω₀ and the absorbed energy and oscillator amplitude is maximized.

An adaptive media, like water, can change its natural frequency ω₀ and the normal state is where the absorbed energy is maximized, i.e. ω₀=ω and the media is then in resonance with the applied energy. Using the classical damped oscillator model results in the following on water containing NaCl; when the applied energy (ω) has high frequency (ex. 10 Hz) a few Na⁺ ions attach to ^{δ-}O of H₂O molecules forming a cluster having the mass m=Σm_{Na}+m_{H2O}, where the host H₂O molecules are part of the mass. A few Cl⁻ ions attach to ^{δ+}H of H₂O molecules forming a cluster having the mass m=Σm_{Cl}+m_{H2O}. The two clusters are connected by a few H₂O molecules and their hydrogen bonds and the sum of the hydrogen bonds constitutes the spring constant k. Each of the clusters have a natural frequency ω₀=√k/m and together they form an oscillating dipole where ω₀=ω. Thus the water is in resonance with the applied energy and the water has a frequency specific molecule structure consisting of clusters of ions. At low frequency (ex. 2 Hz) the clusters of ions are larger, forming a larger mass and the clusters are connected by a longer chain of H₂O molecules with more hydrogen bonds constituting a smaller spring constant k and the natural frequency ω₀=√k/m is lower. The hydrogen bond and its corresponding spring constant k depend on the force between two residual charges and it has a certain size in the longitudinal direction. A small angular twist between two H₂O molecules creates a twist in the hydrogen bond and the “angular” spring constant k_{twist} is much smaller than k. A molecule structure consisting of clusters of ions arranged into oscillating dipoles which create a small angular twist in the hydrogen bond(s) can explain the low resonance frequency of water. The overall water molecule structure can be tetrahedral as reported by Chaplin² and Head-Gordon et al³.

Resonance in water only occurs at extremely subtle energy levels (due to the extremely weak hydrogen bond) and therefore the methods to create resonance and to measure resonance must be arranged accordingly. Resonance in water can be measured and verified using the method described in FIG. 6. Two electrodes (102) and (103) are positioned at some distance (ex. 2m) in the air and connected to a low frequency signal generator (104) (ex. 5 Hz, 500 μV RMS) and this creates conduction current J₁ in the air (typically 0.5 pA at the above parameter setting). J₁ tunnels through most matter with low loss including plastic and glass. A water sample W (ex. 0.1 l water containing 100 ppm NaCl in a glass or plastic container) is positioned (100) within J₁ and then W is moved to a new position (101) a few m away from J₁ and the electrodes. The following happens; the water W creates resonance with the applied electromagnetic energy J₁ and W changes its molecule structure and when W is moved the resonance and molecule structure is maintained by the conduction current J₂, charge in the air constitutes the return path. The amplitude of J₂ is proportional to the amplitude of the resonance in W. This experimental arrangement facilitates measurement of a number of parameters. At 100 ppm NaCl resonance starts at 2 Hz and ceases at 10 Hz as illustrated by graph (105), FIG. 7. When the salt concentration is increased (ex. 5000 ppm NaCl) resonance occurs at lower frequency as illustrated in graph (106), FIG. 7. This is in accordance with the theoretical model; low frequency resonance can only be achieved at large ion clusters, i.e. high salt concentration. When W is heated the frequency range shifts upwards (ex. 4-16 Hz at 85° C.) as illustrated in graph (107). The theoretical model is in large independent of ion or salt type which is confirmed by measurements. The following salts have been investigated and they give similar results (with slight differences in frequency range); sodium chloride, ammonium chloride, phenol-phthalen, copper (II) sulphate, iron (II) sulphate, calcium hydroxide, sodium carbonate, calcium carbonate, ammonium iron (II) sulphate, aluminum potassium sulphate as well as a number of mixtures of these salts and concentrations.

In the above arrangement resonance in W (100 ppm NaCl) starts (in position (100)) at generator amplitude ≧200 μV (or J₁≧0.2 pA) and then resonance is maintained (in position (101)) at significantly lower J₂ (generator amplitude ≧10 μV). This is in accordance with the theoretical model; it takes a certain energy to release ions from existing bonds and to create a new molecule structure, once created it can be maintained at a much lower energy level. When W is in resonance (in position (101)) the generator signal is interrupted for a short time (≈1 s) and then the resonance ceases immediately which is in accordance with the theoretical model; the molecule structure is unstable since the ions are arranged in clusters and when the energy is removed coulomb forces immediately scatter the ions. When W is exposed to electrolyses (ex. 1 μA DC current through W) the resonance ceases and does not regain when the DC current is removed. This is in accordance with the theoretical model; the resonance mechanism consists of clusters of negative and positive ions forming oscillating dipoles and the electrolyses separates the ions towards the cathode and anode. When W is stirred (i.e. ions are evenly distributed) resonance occurs in position (100). W in resonance in position (101) loses its resonance when violently stirred because the molecule structure is changed at mechanical intervention.

W is in position (100) and the generator is tuned to exactly 3.03 Hz (the period 24/2¹⁸ hour). Conduction current J_{Air} with period 24/2¹⁸ hour connects from the air to W and then the generator is switched off; the resonance in W at 3.03 Hz remains for days or weeks and until there is a (short) disturbance where J_{Air} ceases, which sometimes occurs. Tuning the generator to any of the harmonics 24/3·2¹⁷ hour=4.55 Hz, 24/5·2¹⁶ hour=3.79 Hz, 24/7·2¹⁶ hour=5.31 Hz and 24/9·2¹⁶ hour=3.41 Hz give similar results. Thus water can be programmed to create resonance with a specific harmonic of J_{Air} and J_{Air} has sufficient amplitude to maintain the resonance.

Electric fields also create resonance in W. In that case only one electrode (103) is used, the other electrode (102) is connected to ground. W is positioned (100) 0.5 m from the electrode (103) in order to create resonance and then W is moved to position (101) 5 meters away and J₂ between the electrode (103) and W is measured. In this case the resonance is created by electric fields but maintained by conduction current J₂ in the same way as described above. Magnetic fields create resonance in W in a similar way.

### DETAILED THEORY

### Cells

All living species including organisms, that the author has investigated, create resonance with one of the harmonics of the energy in the air J_{Air} and therefore some cells must contain some kind of forced oscillator. The exact nature of this oscillator is not known and it may vary between species. Therefore the proposed mechanism is open for discussion. Nature is good at copying and the mechanism used in the ion channel can explain the oscillator mechanism. In its simplest form, and only as an example, it can consists of two oxygen O⁻ atoms embedded in the cell wall at a distance equal to two H₂O molecules. ^{δ+}H of one H₂O molecule attaches to each O⁻ atom and therefore there are two H₂O molecules locked to the O⁻ atoms and on these two H₂O molecules one Na⁺ ion can attach to the free ^{δ-}O and one Cl⁻ ion can attach to the free ^{δ+}H. This constitutes a forced oscillator having a fixed natural frequency ω₀ (i.e. fixed resonance frequency). A different pattern of embedded atoms create a different molecule structure and resonance period and thus cells can create resonance at the harmonics of J_{Air} having the periods 24/m·2ⁿ hours, m=1, 3, 5, 7 and 9.

Humans have cells containing oscillators embedded in the skull bone, centered in the middle (top) of the skull and the cell oscillators are oriented so that they create resonance with J_{Air} perpendicular to the skull and at the period 24/3·2¹⁷ hour=4.55 Hz. In normal situations conduction current J_{Air} always connects to the top of the skull of a healthy human and it creates resonance in the water contained in the brain fluids and cells and a specific molecule structure is created and the blood circulation distributes this water to the rest of the body. The human body and its water is in resonance with J_{Air} at the period 24/3·2¹⁷ hour. When J_{Air} is removed the water in the body immediately loses its resonance and it takes approx. 20 minutes to regain full resonance amplitude. The following mammals have been investigated and they create resonance in a similar way and the cells containing oscillators are embedded at the top of the skull bone; dogs, cat, sheep, horse, cow, fox, deer, red deer, fallow deer and moose. The location of the cells containing oscillators is easy to find; the skull bone of a dead animal is cut into pieces and the individual pieces are measured with respect to resonance. Very old skulls create resonance as long as some water is added to the top of the skull and dry skulls do not create resonance. A 2000 year old human skull has been tested and it still creates resonance (when water is added). This gives an indication of the nature of the resonance mechanism or oscillator; embedded atoms in the cell wall in combination with ion-water molecules. The skull of an animal can be positioned on top of a container with water W, after a few seconds W is in resonance with J_{Air}, the skull is removed and the resonance in W remains. Reptiles, insects, trees and plants create resonance in a similar way and at the same period. In trees and plants the cells containing oscillators are embedded in the upper part of the root system and that part can be cut into small pieces where the individual pieces create resonance, thus the cells containing oscillators are distributed. Resonance ceases when the root is dried and commences when water is added. There is one exception; birds, crocodiles, alligators and ferns create resonance at 24/2¹⁸ hours (3.03 Hz), obviously because of their biological heritage. Evolution of life changed the resonance mechanism=70 million years ago into something better because it's easier to create high amplitude resonance at the period 24/3·2¹⁷ hour (see FIG. 7).

The spectrum of bacteria is enormous and the author has found bacteria that create resonance (contain oscillators) at the periods 24/2¹⁸ hour (3.03 Hz), 24/3·2¹⁷ hour (4.55 Hz), 24/5·2¹⁶ hour (3.79 Hz), 24/7·2¹⁶ hour (5.31 Hz) and 24/9·2¹⁵ hour (3.41 Hz). For instance a piece of fouled meat or polluted water contains a broad spectrum of bacteria that create resonance at all of the above periods. As an example the bacteria Lactobacillus acidophilus, Lactobacillus plantarum 299v and Shigella sonnei create resonance at the period 24/5·2¹⁶ hour (3.79 Hz). When a bacteria culture (ex. Lactobacillus acidophilus or Lactobacillus plantarum 299v) is positioned in water, J_{Air} also propagates into the water and creates resonance in the water and the water molecule structure is changed. Thus bacteria indirectly change the molecule structure of water. A few types of viruses (ex. herpes) have been investigated and they create resonance at the period 24/5·2¹⁶ hour (3.79 Hz). Two type of cancer (malignant tumors) have been investigated, they create resonance at the period 24/5·2¹⁶ hour (3.79 Hz).

The following is observed. A healthy person always creates resonance at the period 24/3·2¹⁷ hour (435 Hz) and the resonance amplitude is relatively large which can be measured by means of J_{Air} (typically 0.2-0.5 pA) that connects to the person's skull. When the person has a pathogenic bacteria or virus infection J_{Air} with different period (usually 24/5·2¹⁶ hour (3.79 Hz)) connects to the infected area and the amplitude increases at serious infections and at the same time the amplitude of resonance in the body water decreases and at serious infections, inflammations and malignant tumors the resonance at the period 24/3·2¹⁷ hour (4.55 Hz) ceases completely. The resonance at the period 24/3·2¹⁷ hour (4.55 Hz) gradually regains its amplitude as the person cures.

An interesting observation is that beneficial bacteria (bowl), which live in symbiosis, create resonance at the period 24/3·2¹⁷ hour (4.55 Hz), i.e. the same period as the host.

### DETAILED THEORY

### Air

Interaction between low frequency displacement current ∝D/∝t, the geomagnetic field and electrons in the air creates a low frequency resonance phenomenon in the air and conduction current J_{Air} with the basic period 24 hours and harmonics with periods 24/m·2ⁿ hours. The earth's geomagnetic fields rotate in the sun wind and this creates conduction current with 24 hours period, probably in the magnetosphere and it probably has the same origin as the Birkeland current described by Potemra¹¹ or perhaps the origin is the ionosphere Sq current where the Sq current is described by Aronson et al.¹², Maus and Lühr¹³ and Sabaka et al.¹⁴. The conduction current creates displacement current according to Maxwell's equation curl H=J+∝D/dt. ∝D/∝t is almost static and can therefore be measured using the following method. Transversal electromagnetic waves ∝D/∝t having electric field vectors E_{FV} that propagate through a media having charge density ρ creates an electric body force ρE_{FV} on the media and there is a corresponding back reaction force on E_{FV} and this gives ∝D/∝t a change of impulse as reported by Bostrom and Fahleson¹⁵ and Melrose and McPhedran¹. An electrode, positioned in the air, and connected to a voltage source U creates a charge −q in the air as illustrated in FIG. 8. This creates a back reaction force on E_{FV} and change of impulse of ∝D/∝t and waves of ∝D/∝t propagates towards −q and since ∝D/∝t is coherent and polarized the individual field vectors add to much larger (amplified) electric field vectors E and magnetic field vectors B close to the charge and they can be measured with E- or B-field probes. The measured values are (in Stockholm, Sweden); E_{FV}=3-10 V/m, B_{FV}=10-30 nT and the nodes occur at exactly 00¹³ and 12¹³ (GMT+1 h). ∝D/∝t is almost square wave shaped and thus it contains large amount of harmonics. E_{FV} and B_{FV} can be amplified approx. 50 times at U=50-100 V. This measurement method can of course only be used on almost static ∝D/∝t. Being almost static ∝D/∝t can normally only propagate in one way; parallel to the geomagnetic field B_{GM} and the magnetic field vectors B_{FV} are directed north-south in the vertical plane because then the labor B_{GM}·B_{FV}=0. As a consequence the orthogonal electric field vectors E_{FV} are directed east-west in the horizontal plane.

The global electric circuit describes how lightning transfers electrons to the earth crust and how the 200 kV potential between the atmosphere and earth crust transfers the charge back to the atmosphere and thus the air contains ≈500 pC/m³ electrons as reported by Israel¹⁶, Israelsson et al.¹⁷, Roble¹⁸ and Rycroft et al.¹⁹. ∝D/∝t creates an electric body force ρE_{FV} on the electrons in the air and it makes the electrons drift in an oscillating motion and create conduction current J_{Air} in the air. The electrons in the air can accumulate (−q) and deplete (q) and drift (J_{Air}). Two charges −q/q at some distance with J_{Air} in between is a forced oscillator with time constant q/J and when the period of the applied energy ∝D/∝t equals q/J (or harmonics) the forced oscillator oscillates (is in resonance). Air is an adaptive media; its charge has large freedom in distribution. An adaptive media strives to maximize the absorbed energy, i.e. the oscillators adapt their charge so that they create resonance with applied energy. The global electric circuit supplies the air and the oscillators with charge until q/J has a value that creates resonance and from that point on surplus charge is transferred to the atmosphere. It's self regulating.

In order to understand how such oscillators can be stable in the air the following experiment is performed. Two electrodes, connected to a voltage source, are positioned at some distance (ex. 2 m) in the air. This creates a dipole −q/q as illustrated in FIG. 9. Both charges create a back reaction force on ∝D/∝t and thus ∝D/∝t propagate towards −q and then along the dipole towards q and then ∝D/∝t resume their original directions and close to the dipole the electric and magnetic field vectors are amplified, i.e. E and B. There is a force (B·B_{GM}) between B and the geomagnetic field B_{GM} and this diverts an extremely small part of B_{GM} along the dipole and this magnetic field is called ΔB_{GM}. Electrons propagate from −q to q, i.e. conduction current J. A magnetic field ΔB_{GM} creates a force F=−ev_{c}×ΔB_{GM} on an electron with charge −e, mass m and speed v_{c} and it makes the electron propagate in a circle with radius r=mv_{c}/eΔB_{GM}. The electron also has speed along the dipole so the total motion is a helix. Typical values are; drift velocity of electrons in air=20 m/s, radius of the helix r=7 cm, speed forward (along the dipole)=6 m/s and thus the diverted magnetic field ΔB_{GM}=1.5 nT compared to the geomagnetic field ≈50 μT. Thus electrons and J propagate in a controlled way within dipoles in the air. In the next experiment the voltage source and the electrodes are removed; the dipole hovers in the air and the body force from ∝D/∝t turns the dipole slowly into a stable horizontal, east-west position and ΔB_{GM} creates stable J within the dipole. The hovering dipole remains stable until J has leveled out the two charges which takes typically 20 minutes at U=10 V (i.e. −g≈300 pC). Thus forced oscillators consisting of −q/q and J_{Air} are stable because of the subtle balance between ∝D/∝t, the geomagnetic field and electron charge and mass.

Thus electromagnetic waves of conduction current in air can only exist in the presence of the geomagnetic field. A constant magnetic field, ≧10 μT, for instance created by a permanent magnet (see FIG. 9) within or close to the conduction current increases, decreases or extinct the magnetic field, B_{GM}, and this increases, decreases or eliminate J_{Air} depending of polarity and amplitude of the magnetic field. This method can be used to influence J_{Air}.

Being an adaptive media air strives to organize the oscillators so that the absorbed energy is maximized; the oscillators are organized into a 3D matrix where the oscillators are mutually coupled. The 3D matrix has a logical structure and it consists of only one repetitive basic element; four charges are organized into a horizontal quadrant as illustrated in FIG. 10. Two charges, −q and q (109), and conduction current J_{Air} (108) between the charges constitutes one oscillator. Thus the four charges constitute four oscillators (mutually coupled and oscillating in two dimensions). The starting point of the 3D matrix is one quadrant that oscillates at the period 24 hours, consisting of a quadrant of four oscillators at the period 24 hours, each denoted 24₁ in FIG. 11. This quadrant is divided into four new quadrants, each containing four oscillators, denoted 24₂, and which oscillate at the period 24/2=12 hours. Each of these quadrants is divided into four new quadrants, each containing four oscillators, denoted 24₄, and which oscillate at the period 24/4=6 hours and so it continues until a fine meshed grid is created because the process is non linear. This pattern is repeated in all four geographical directions forming a large 2D grid as illustrated in FIG. 11. FIG. 11 shall be interpreted in the following way. The “line” denoted 24₁ consists of a long chain of oscillators (q, −q, q, −q, q etc.) at the period 24/1 hours and super positioned there are oscillators at the periods 24/2, 24/4 etc. hours; altogether 24₁ consists of a chain of super positioned oscillators and super positioned conduction currents having the periods Σ24/2ⁿ, n=0, 1, 2, 3, 4, . . . hours. 24₂ consists of a chain of super positioned oscillators and super positioned conduction currents having the periods Σ24/2ⁿ, n=1, 2, 3, 4, . . . hours, and so it continues. Thus the electrons in J_{Air} only have to bridge the distance of the shortest oscillator/dipole in the chain and that explains why conduction current having extremely long period can be transported in a stable way in the air. The air contains one horizontally positioned 2D grid approx. 1 meter above ground and then every 3 meter there is another (phase shifted) horizontal 2D grid. FIG. 12 illustrates how layers of 2D grids (110) form a 3D matrix (111). In those nodes where the energy or amplitude is sufficiently high there is also resonance in the vertical plane, thus the horizontal 2D grids are interconnected (and mutually stabilized) by vertical oscillators and conduction current J_{Air} (112). Thus the 3D matrix oscillates in 3 dimensions.

The first odd harmonic having the period 24/3=8 hours creates a similar 2D grid oriented east-west (i.e. 45 degrees to the above 2D grid) having oscillators at the periods 24/3·2ⁿ hours and since the two grids emanate from ∝D/∝t they are in phase in some nodes, theses nodes contain vertical oscillators (that oscillate at the periods 24/2ⁿ and 24/3·2ⁿ hours) and vertical J_{Air} (112) and therefore the two grids are linked by mutual resonance creating a common 3D matrix. The 3D matrix also contains oscillators and J_{Air} at the periods 24/5·2ⁿ, 24/7·2ⁿ and 24/9·2ⁿ hours. Thus the 3D matrix oscillates in three dimensions and in many modes. It's a complex but logical structure.

The amount of harmonics in the 3D matrix is very large. Thus J_{Air} contains harmonics having the following periods/frequencies; 24/2¹⁸ hour=3.03 Hz, 24/3·2¹⁷ hour=4.55 Hz, 24/5·2¹⁶ hour=3.79 Hz, 24/7·2¹⁶ hour=5.31 Hz and 24/9·2¹⁵ hour=3.41 Hz. These periods/frequencies coincides with the frequency range where water creates resonance.

### DETAILED THEORY

### Conduction Current and Resonance in Conductors

Current normally propagates in a conductor with almost the speed of light. There are two mechanisms involved in this process. Electrons in a conductor drift with their drift velocity, in copper the drift velocity is ≈1.5 mm/s. Injected electrons create a charge gradient and the coulomb forces of this gradient act on nearby electrons and this creates a chain reaction which propagates with almost the same speed as the coulomb forces, i.e. the current propagates with almost the speed of light. Current transport is conducted by electrons on the conductor surface, called surface charge. When the injected energy is extremely small, ≦1 pA, the gradient is also extremely small. The surface charge attracts positive ions in the air and the number of positive ions is sufficient to neutralize the gradient of the surface charge, i.e. the coulomb forces are neutralized. Thus the current propagates with the drift velocity of the electrons, i.e. ≈1.5 mm/s. A conductor submerged in water behaves in the same way since positive ions in the water neutralize the surface charge. The exact current speed in copper is measured to 1.52 mm/s. The wave propagation in a conductor at current ≦1 pA is described by the classical wave propagation in one dimension. One solution is a harmonic wave that satisfies ξ=a·sin(wt+δ), where a is the amplitude, δ is the phase constant and ω is the angular frequency. The wave velocity is equal to the velocity v of the current, i.e. the drift velocity of the electrons, v=1.52 mm/s in copper. Resonance and a standing wave inside the conductor occur when its length is equal to one half wavelength (λ/2). A conductor with certain length L creates a standing wave and resonance at L=λ/2=v·T/2, where T is the period. This constitutes a resonance circuit or (forced) oscillator. Such resonance circuits or oscillators are utilized to create resonance with J_{Air}, and they are utilized in apparatus in order to generate conduction current having specific period. When L=λ/2=v·24·60·60/m·2·2ⁿ the oscillator creates resonance with one of the harmonics of J_{Air}. Resonance at period 24/3·2¹⁷ hour is achieved at L=v·24·60·60/3·2·2¹⁷ m. However an even multiple of L can also be used since J_{Air} always contains super positioned even harmonics. The amplitude of the resonance can be increased by connecting the oscillator to a sinus signal (ex. 1V RMS, period 24/3·2¹⁷ hour=4.55 Hz). The oscillator is connected, through a wire, capacitor or electric fields to a signal generator.

### DETAILED THEORY

### Conditioning

Resonance in water is created when electromagnetic energy is injected and ions are released from their existing bonds and arranged into new molecule structures. The applied energy is extremely small and empirically it has been found that only a part of the ions are arranged into new molecule structures and therefore the amplitude of the resonance is limited to approx. 25% of the theoretical maximum value. A mechanical pulse releases ions from their bonds and allow them to move to new molecule structures. This pulse can for instance consist of a sound wave with frequency 0.1-5 kHz and duration 1-10 s. Such procedure increases the resonance amplitude by approx. 100%.

A similar effect is achieved when the water is brought to resonance at the period 24/2¹⁸ hour=3.03 Hz for a few seconds and then to resonance at the period 24/3·2¹⁷ hour=4.55 Hz. The empiric result is approx. 50% increase in resonance amplitude at the period 24/3·2¹⁷ hour.

A similar effect is achieved when water is simultaneously exposed to conduction current having the period 24/3·2¹⁷ hour=4.55 Hz and the period 24/2¹⁸ hour=3.03 Hz and in particular conduction current with the period 24/3·2¹⁷ hour and two waves with the period 24/2¹⁸ hour having 180 degrees phase difference. The conduction current J_{Air} contains very long periods and those components make the positive and negative ions drift apart (similar to electrolyses) and this negative influence is minimized when the water is exposed to waves having 180 degrees phase difference. In the latter case the increase in resonance amplitude is approx. 100% and it can be combined with mechanical sound waves in which case the total increase in resonance amplitude is approx. 200%. Maximum Q-value of water occurs at approx. 4.5 Hz and therefore water strives to create resonance at the period 24/3·2¹⁷ hour=4.55 Hz and energy at other periods releases the ions from bonds in the previous molecule structures so that they can move to new structures.

### DETAILED THEORY

### Measurement Technique

The 3D matrix in the air consists of oscillating charges −q/q and conduction current J_{Air}. q is typically 10-1000 pC and J_{Air}≦1 pA. J_{Air} contains extremely low frequency components, i.e. they are almost static. Thus the laws of electrostatics can be applied. External charge, DC current, static electric- and magnetic fields and also the almost static ∝D/∝t can be used to influence q and J_{Air} and this creates body force and back reaction force and change of impulse according to the laws of electrostatics and electrodynamics and this can be measured as a change in position or direction of the applied energy or q or J_{Air}. This enables a number of non correlated measurements of q and J_{Air} (giving the same results). The following instrument can be used. J_{Air} can be described as a distributed charge qₖ. A charge qₚ positioned at a distance r is exposed to the force F from J_{Air}; F=Σqₚqₖrₖ/4πεₒrₖ³. A short wire, called a probe, is utilized to make an instrument. The probe is moved with constant speed through J_{Air} or q. The charge qₚ in the instrument probe is influenced by the force F which generates a current pulse. The probe is connected to a high gain JFET amplifier, the current pulse is amplified, band pass filtered to suppress DC and AC noise and displayed. In this way the position and amplitude of q and J_{Air} are measured. The conduction currents J₁ and J₂ in FIG. 1 are measured in the same way.

### DETAILED THEORY

### Conclusions

There is a reason that so many different types of species including organisms (i.e. all species that the inventor has investigated with no exception) create resonance and absorb low frequency energy present in the air. Advanced life forms create resonance at the 24/3·2¹⁷ hour (4.55 Hz) while pathogens such as pathogenic bacteria, pathogenic viruses and cancer but also mould and fungus create resonance at another period, usually 24/5·2¹⁶ hour (3.79 Hz). Evolution of life has developed some biological processes where the processes are controlled or influenced by specific water molecule structures. Therefore advanced life forms have cells containing oscillators embedded at a strategic place (ex. skull bone) which certifies that its water always is in resonance with J_{Air} and that the proper molecule structures are maintained. Disease-causing organisms, i.e. pathogens, contain oscillators because of the same reason, but at a different period since that enables the pathogenic bacteria, pathogenic viruses, cancer or fungal microbes to disturb biological processes of the host and this enhances their chance of survival. They use electromagnetic warfare.

Studies couple electromagnetic fields to cell biology as reported by Adams et al.⁴ and Zhao et al.⁵. Electric fields produced by ion channels (especially H⁺, K⁺ and Ca²⁺) provide specific signals that regulate cell behavior during embryonic development, normal tissue turnover and regenerative repair as reported by Levin⁶. The ion channel contains a matrix of oxygen, O⁻, atoms where the shape of the matrix matches the shape of a certain ion-H₂O molecule as reported by MacKinnon et al.^{7,8,9}. The water channel builds on a similar mechanism as reported by Agre et al.¹⁰.

The following theory is proposed. The water in the human body has certain molecule structures, based on resonance at the period 24/3·2¹⁷ hour (4.55 Hz). Some biological processes such as the blood-brain-barrier, receptors and ion channels depend on certain and stable water molecule structures. Pathogenic bacteria, viruses and cancer create resonance at a different period and this change the water molecule structures in the vicinity of the pathogens or the illness. Blood is pumped into the area and the water at the border is constantly subjected to change in molecule structures and thus ion channels and receptors are exposed to a variety of molecule structures and free ions which disturb the processes the ion channels and receptors are programmed to perform. For instance free positive ions can attach close to the ion channel wall and neutralize the charge of the O⁻ atoms (in the ion channel). Receptors of type ligand-gated ion channels have a gating mechanism that can be disturbed in a similar way. The phenomenon is similar to Debye shielding and it's likely to change the behavior of the ion channel and the gating mechanism. A similar phenomenon is known as the Gouy-Chapman theory which describes the effect of double layer static surface charge on the cell membrane potential.

Thus part of the human or animal body (and other species) can contain water which has resonance at a period different to the period 24/3·2¹⁷ hours (caused by pathogens) or no resonance at all and this disturbs some biological processes. Injecting electromagnetic energy having the period 24/3·2¹⁷ hour (and the period 24/2¹⁸ hour) creates resonance in the water and it improves said biological processes. Repeating the procedure ensures that the resonance at the period 24/3·2¹⁷ hour is maintained.

Injecting electromagnetic energy into organisms (including pathogens) like bacteria, viruses, cancer, mould and fungal microbes creates resonance in their water and when the period of said energy deviates from said organisms natural resonance period it disturbs biological processes in said organisms. Prolonged exposure creates permanent damage and said organisms permanently lose their ability to create resonance with the energy in the air and thus pathogens lose their ability to electromagnetically influence the host. Injecting energy with the period 24/3·2¹⁷ hour (and the period 24/2¹⁸ hour) is beneficial because it harms pathogens and at the same time it influences the host in a positive way as well as beneficial bacteria that live in symbioses.

Persons in bad health condition, example hospitalized and immobilized persons, often have low resonance amplitude in their water. Injecting electromagnetic energy at regular intervals at the period 24/3·2¹⁷ hour increases the amplitude of the resonance in their water and this improves their overall health condition.

The human taste depends on receptors which are influenced by water having resonance at the same period as the human body, i.e. the period 24/3·2¹⁷ hour. The taste experience depends on how well consumed water in food or beverages matches the resonance and water molecule structures of the receptors. Therefore food and beverages that have resonance in the water at the period 24/3·2¹⁷ hour taste better.

In nature food normally has resonance in its water at the period 24/3·2¹⁷ hour. However when food is processed and transported the resonance is lost. Food and beverages consumed, that does not have resonance in their water, decreases the body's resonance amplitude with approx. 10-25% at the period 24/3·2¹⁷ hour, since part of the energy is used to create resonance in the consumed water. In contrary food and beverages consumed, that has resonance in its water at the period 24/3·2¹⁷ hours, maintains and sometimes increases the body's resonance amplitude (approx. 10%) which can be beneficial from a well being point of view. A glass of mineral water (large content of ions), which is in resonance at the period 24/3·2¹⁷ hour, can significantly increase the amplitude of the resonance in a hospitalized person's water (≈50-100%).

The overall conclusion is that advanced species, including humans contain water that has resonance at the period 24/3·2¹⁷ hour and the resonance is maintained by J_{Air} and this creates specific water molecule structures which is important to a number of biological processes. The resonance and the corresponding molecule structures in water contained in humans and other species or in water consumed can be disturbed in a number of ways. The object of the invention is to create resonance that is maintained by J_{Air} in said water at the period 24/3·2¹⁷ hour in order to improve biological processes that are related to resonance in water and water molecule structures. This is performed by exposing the water to electromagnetic energy having the period 24/3·2¹⁷ hour and the period 24/2¹⁸ hour and this procedure also destroys the ability of organisms (including pathogens) like bacteria, viruses, cancer, mould and fungal microbes to create resonance with J_{Air} at their own natural period.

### DETAILED THEORY

### References

1. Melrose D. B, McPhedran R. C. Electromagnetic processes in dispersive media. Cambridge University Press, Cambridge, pp 106-110 (1991).
2. Chaplin M. A proposal for structuring of water. Biophysical Chemistry. 83, 211-221 (1999).
3. Head-Gordon T, Johnsson M. E. Tetrahedral structure or chains for liquid water. Available online http://www.pnas.org/cgi/doi/10.1073/pnas.0510593103 (2006).
4. Adams D. S, Masi A, Levin M. H+ pump-dependant changes in membrane voltage, are an early mechanism necessary and sufficient to induce Xenopus tail regeneration. Development. 134, 1323-35 (2007).
5. Zhao M, Dick A, Forrester J. V, McCaig C. D. Electric field-directed cell mobility up-regulated expression and asymmetric redistribution of the epidermal growth factor receptors and is enhanced by fibronectin and laminin. Mol Biol Cell. 10, 1259-76 (1999).
6. Levin M. Large-scale biophysics: ion flows and regeneration. Trends Cell Biology. 17, 261-70 (2007).
7. MacKinnon R, Morais-Cabra J. H, Zhou Y, Mann S. Potassium channel receptor site for the inactivation gate and quaternary amino inhibitors. Nature. 411, 657-661 (2001).
8. MacKinnon R, Morais-Cabra J. H, Zhou Y. Energetic optimization of ion conduction rate by K⁺ selectivity filter. Nature. 414, 37-42 (2001).
9. MacKinnon R, Morais-Cabra J. H, Zhou Y, Kaufman A. Chemistry of ion coordination and hydration revealed by a K⁺ channel-Fab complex at 2.0 Å resolution. Nature. 414, 43-48 (2001).
10. Agre P, Pohl P, Saparov S. M, Borgnina M. J. Highly selective water channel activity measured by voltage clamp: Analysis of planar lipid bilayers reconstituted with purified AqpZ. Available online http://www.pnas.org/content/98/17/9624.full (2001).
11. Potemra T. A. Observation of Birkeland currents with the TRIAD satellite. Astrophysics and Space Science. 58, 207-226 (1978).
12. Aronson E. B, Ware R. H, Bender P. L. Modelling of solar quiet magnetic field variations near a conductivity anomaly. Geophysical Journal International. 59, 539-552 (1979).
13. Maus S, Lühr H. Signature or the quiet-time magnetospheric magnetic field and its electromagnetic induction in the rotating Earth. Geophysical Journal International. 162, 755-763 (2005).
14. Sabaka T. J, Olsen N, Langel R. A. A comprehensive model of quiet-time, near-Earth magnetic field: phase 3. Geophysical Journal International. 151, 32-64 (2002).
15. Bostrom R, Fahleson U. Vertical propagation of time-dependant electric fields in the atmosphere and ionosphere. Proc. Fifth Int. Conf. Atmospheric Electricity, 2-7 September, pp 529-534 (1974).
16. Israel H. Atmospheric Electricity. U.S. Department of Commerce, Springfield (1973).
17. Israelsson S, Knudsen E, Anisimov S. V. Vertical profiles of electrical conductivity in the lowermost part of the turbulent boundary layer over flat ground. Journal of Atmospheric and Terrestrial Physics. 56, No. 12: 1545-1550 (1994).
18. Roble R. G. On modelling component processes in the Earth's global electric circuit. Journal of Atmospheric and Terrestrial Physics. 53, 831-847 (1985).
19. Rycroft M. J, Israelsson S, Price C. The global atmospheric electrical circuit, solar activity and climate change. Journal of Atmospheric and Solar-Terrestrial Physics. 62, 1563-1576 (2000).

### BRIEF DESCRIPTION OF THE DRAWINGS

The invention will be described in more detail in the following, with reference to the appended drawings, in which:

FIG. 1 describes a method to create resonance in water using passive oscillators.

FIG. 2 describes a method to create resonance in water using active oscillators.

FIG. 3 describes a method to create resonance in water using electric fields.

FIG. 4 describes a method to create resonance in water using magnetic fields.

FIG. 5 describes a method to create resonance in water using conduction current.

FIG. 6 describes the experimental arrangement to create resonance in water.

FIG. 7 describes the amplitude of resonance in water as a function of frequency.

FIG. 8 describes electromagnetic waves of displacement current with 24 hours period.

FIG. 9 describes an electric dipole in the air.

FIG. 10 describes the resonance mechanism of conduction current in the air.

FIG. 11 describes the forming of even harmonics of conduction current in the air.

FIG. 12 describes how 2D grids of conduction current form a 3D matrix.

### DESCRIPTION OF THE INVENTION

The object of this invention is to create a method which creates resonance in water at the period 24/3·2¹⁷ hour and when the resonance has been created said resonance is maintained by electromagnetic energy in the air having the period 24/3·2¹⁷ hour.

The method is to expose water to electromagnetic energy having the period 24/3·2¹⁷ hour. The amplitude of this energy must be higher than the amplitude of the corresponding energy in the air. Said electromagnetic energy can consist of electric fields, magnetic fields, conduction current or combinations of said electromagnetic energies. Small volumes of water (ex. the human body) are normally exposed a short time (10-100 s) and large volumes (ex. a water reservoir) can be exposed a longer time. Resonance in water is created at very small energy level and the level must be adapted to the water volume. At small water volumes (ex. the human body) resonance can be achieved at the following (indicative) levels; conduction current ≧0.5 pA, electric fields ≧5 mV/m, magnetic fields ≧50 nT.

Resonance at the period 24/3·2¹⁷ hour relates to specific water molecule structures. Thus ions must be released from existing bonds and transferred to new molecule structures. This process is improved when water, in addition to energy at the period 24/3·2¹⁷ hour, is exposed to electromagnetic energy with a deviating period and this energy releases the ions from bonds and enables them to move to new molecule structures creating resonance at the period 24/3·2¹⁷ hour. One method used is to expose water to electric- or magnetic fields having the period 24/2¹⁸ hour and/or one electromagnetic wave of conduction current having the period 24/2¹⁸ hour or two electromagnetic waves of conduction current having the period 24/2¹⁸ hour and 180 degrees phase difference. The water can, in a first step, be exposed to energy having the period 24/2¹⁸ hour and in a second step to energy having the period 24/3·2¹⁷ hour alternatively performing the two steps simultaneously. The second method used is to expose water to mechanical waves, preferably sound waves having preferably the frequency f, 0.1 kHz≦f≦5 kHz during the time t, 1≦t≦10 s. Said methods are called conditioning and they can be combined.

The invention encompass all water including sea water, lake water, river water, water in wells, water in reservoirs, water in beverages, water in food, water in human body fluids and cells, water in other species and organisms and molecule structures consisting of H₂O molecules and ions.

The object of this invention is also to create methods which makes it possible to limit and/or to destroy the ability of organisms like bacteria, viruses, cancer, mould and fungal microbes to create resonance with electromagnetic energy in the air having the periods 24/2¹⁸ hour, 24/3·2¹⁷ hour, 24/5·2¹⁶ hour, 24/7·2¹⁶ hour and 24/9·2¹⁵ hour. Destroying or limiting the ability to create resonance limits the probability of survival of said organisms. Disease-causing organisms, i.e. pathogens, create resonance at one of the periods 24/5·2¹⁶ hour, 24/7·2¹⁶ hour and 24/9·2¹⁵ hour. The method used is to expose said pathogens to electromagnetic energy having the period 24/3·2¹⁷ hour and the period 24/2¹⁸ hour (according to the above methods) which is preferably used in vivo and in vitro. The method has the advantage that the electromagnetic energy does not harm the human and beneficial bacteria that live in symbioses. The exposure time is preferably 30 s, 3 times daily in 10 days and depends on type of pathogen, seriousness of the infection and general condition of the human. An alternative method can be used where organisms (including pathogens) are exposed to electromagnetic energy having the frequency f, 1 Hz≦f≦15 Hz, preferably 3 Hz≦f≦6 Hz, but with a period different to the natural resonance period of said species, i.e. a period deviating ≧+/−5% from the periods 24/2¹⁸ hour, 24/3·2¹⁷ hour, 24/5·2¹⁶ hour, 24/7·2¹⁶ hour and 24/9·2¹⁵ hour. The exposure time is typically 24-96 hours but can also be continuous depending on application. This procedure is preferably used in vitro and when the spectrum of said organisms is broad (ex. a large number of different types of bacteria creating resonance at many harmonics including the period 24/2¹⁸ hour and the period 24/3·2¹⁷ hour).

The invention encompasses all types of organisms including all types of types of disease-causing pathogens causing infections and inflammations, all types of bacteria, all types of viruses, all types of cancer cells and tumors, all types of mould and all types of fungal microbes.

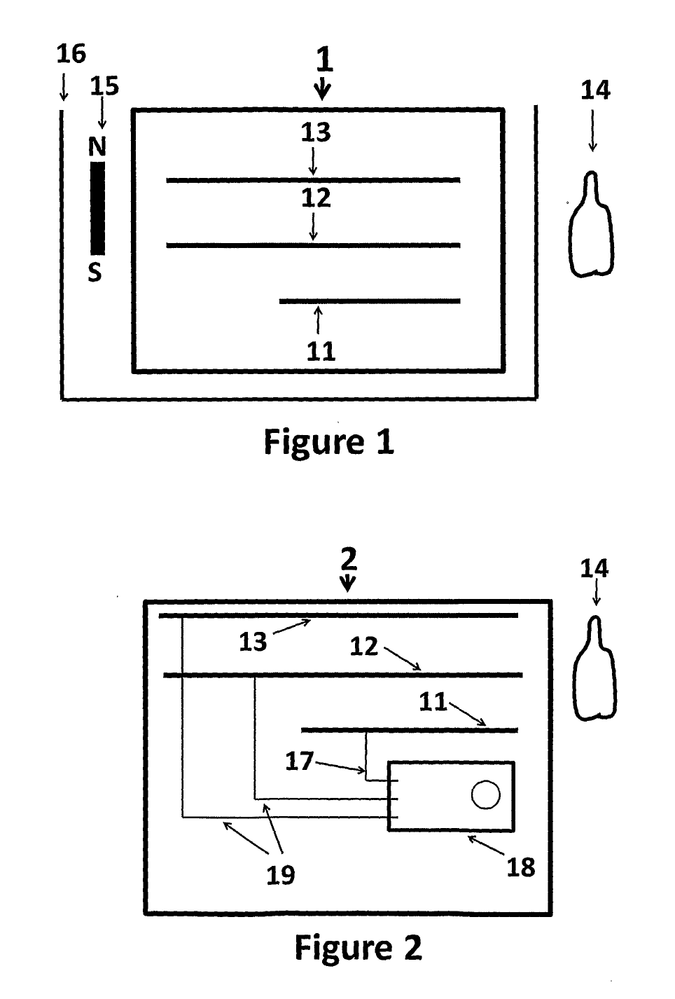

The invention includes a number of methods to generate electromagnetic waves of conduction current, electric fields and magnetic fields as described in FIGS. 1-5.

FIG. 1 describes a method to create conduction current utilizing (passive) resonance circuits also called (passive) oscillators. Each oscillator consists of a conductor (11), (12) and (13) with a specific length that creates resonance. Oscillator (11) creates resonance at the period 24/3·2¹⁷ hour or an even multiple, ex. the period 24/3·2¹⁴ hour (since J_{Air} contains super positioned waves of conduction current having even harmonics). The length of the conductor is L=λ/2=v·T/2 where T is the period of the desired resonance and v is the wave velocity in the conductor (v=1.52 mm/s in copper). As an example an oscillator at the period 24/3·2¹⁴ hours has conductor length

L₃=1.52·10⁻³·24·60·60/3·2·2¹⁴=1.34·10⁻³m=1.34 mm.

Electromagnetic waves of conduction current in the air J_{Air} with super positioned periods Σ24/3·2ⁿ hours and including the period 24/3·2¹⁷ hour connect to the oscillator, propagates through it, exit and then connect to another J_{Air} in the air. Thus the oscillator diverts electromagnetic waves of conduction current J_{Air} in the air with certain period content to a specific position. The amplitude of the conduction current depends on the Q-value of the oscillator and how well it's tuned to the period of J_{Air}. The object containing water (14) is positioned in front of the oscillator and in the middle of the conduction current. The conduction current propagates through the water and creates resonance at the period 24/3·2¹⁷ hour. As an option one (12) or two (12 & 13) oscillators having lengths that creates resonance at the period 24/2¹⁸ hour or an even multiple can be included in order to condition the water. When two oscillators (12 & 13) are positioned in parallel at a small distance (≈2-100 mm) they automatically oscillate with 180 degrees phase difference. The oscillator (11) (the period 24/3·2¹⁷ hour) has conductor length L₃=v·24·60·60/3·2·2ⁿ, 10≦n≦17, and preferably n=17. The oscillators (12, 13) (the period 24/2¹⁸ hour) have conductor lengths L₁=v·24·60·60/2·2ⁿ, 10≦n≦18, and preferably n=18. The oscillators can preferably be implemented on a printed circuit board or as a thick film device or on a substrate as integrated circuit and they can be very small (length of oscillators ≈200 μm and positioned in parallel at a small distance ≈1 mm). The oscillators can be housed in a container (1) containing the oscillator (11) and as options oscillators (11, 12, 13), oscillator (12), oscillators (12, 13) and oscillators (11, 12). Positive ions must be present close to the oscillators in order to ensure the correct conduction mechanism. This can be achieved in different ways. The container (1) can be filled with (salt) water which ensures that positive ions (ex. Na⁺) are always present. Alternatively the container (1) can be provided with holes ensuring that air or water circulates within the container. The container (1) and the oscillators can be used in air or submerged into water. This method allows injection of conduction current into a small, well specified area and thus the injected energy can be concentrated and limited to the part of the body that is infected. It's also beneficial to the patient that the injected energy is in phase with J_{Air}, because it minimizes electromagnetic interference.

FIG. 1 also describes a method to increase, decrease or extinct electromagnetic waves of conduction current in the air by influencing the geomagnetic field with a constant magnetic field (15), preferably created by a permanent magnet positioned so that said constant magnetic field influences or extinct the geomagnetic field within or close to said electromagnetic waves of conduction current. Said conduction current can only exist in the presence of the geomagnetic field. When the container (1) and/or oscillators (11, 12, 13) are not in use it can be beneficial if the oscillators do not create conduction current. The container (1) and/or said oscillators is then placed in an a container (16) having a device (15) that produces a constant magnetic field, ≧10 μT, which extinct the geomagnetic field, preferably a permanent magnet, and this eliminates creation of conduction current.

FIG. 2 describes a method to create conduction current using active oscillators utilizing (passive) oscillators (11, 12, 13) according to the method described in connection with FIG. 1. The oscillator (11) is connected, through a wire (17), a capacitor or electric fields to a (sinus) signal generator (18) having the period 24/3·2¹⁷ hour, ≦+/−1%, and the amplitude U, 0 V≦U≦100 V RMS. This arrangement increases the amplitude of the conduction current at the period 24/3·2¹⁷ hour in the oscillator. As option oscillators (12, 13) can be included and connected, through wires (19), capacitors or electric fields to the signal generator (18) which has two 180 degrees phase shifted outputs with the period 24/2¹⁸ hour, ≦5+/−1%, and the amplitude U, 0 V≦U≦100 V RMS. Preferably the generator (18) generates the period 24/3·2¹⁷ hour and two 180 degrees phase shifted signals having the period 24/2¹⁸ hour derived from the same clock. The conduction current is proportional to the generator output voltage and can thus be varied depending on application. The oscillators can be integrated together with the generator (18) as a thick film device or as an integrated circuit. The oscillators and the generator can be housed in a container (2) and the oscillators are housed in an environment containing positive ions as described in connection with FIG. 1. The container (2) and/or the oscillators and generator can be placed in a container (16) having a magnetic device (15) as described in connection with FIG. 1). Said generator can be connected to a battery and/or to control means which controls amplitude, period, phase, sequence and ON/OFF.

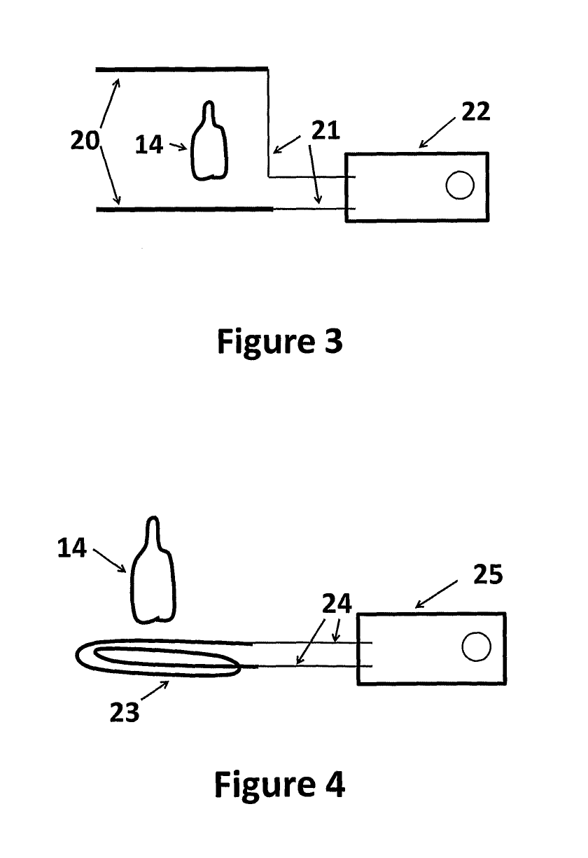

FIG. 3 describes a method by which resonance in water (14) is created by electric fields. One, two (or more) electrodes or plates (20) are positioned in the air and connected, through wires (21), to a signal generator (22) having the period 24/3·2¹⁷ hour, ≦+/−1%. The configuration and generator amplitude depends on the application. The method can be used to create resonance in a small part of the human body. In that case a small electrode is positioned close to the area and the signal generator output voltage U is typically 0.2 mV≦U≦100 mV RMS and the other electrode has galvanic connection to the body or is grounded, in which case charge in the air constitutes the return path. A large sample of water (ex. a large batch of food) demands a different configuration consisting of many electrodes spread over a larger area and the signal generator output voltage U can be 1V≦U≦100 V RMS. Conditioning can, as option, be achieved by super positioning energy having the period 24/2¹⁸ hours, ≦+/−1%, and in that case the signal generator (22) has one output containing two added signals (the period 24/3·2¹⁷ hour+the period 24/2¹⁸ hour). Conditioning can also be achieved by sequential use of the two energies, water is in a first step exposed to the period 24/2¹⁸ hour, preferably during the time t, 10 s≦t≦100 s and in a second step to the period 24/3·2¹⁷ hour, preferably during the time t, 10 s≦t≦100 s. Said generator can be connected to control means which controls amplitude, period, phase, sequence and ON/OFF.

FIG. 4 describes a method by which resonance in water is created by magnetic fields. One (or many) coils (23) (ex. Helmholtz coils) are connected, trough wires (24) to a (current) signal generator (25) having the period 24/3·2¹⁷ hour, ≦5+/−1%. Depending on application the magnetic field B is typically 10 nT≦B≦100 nT (ex. when used on a small are of the human body) or 0.1 μT≦B≦10 μT (ex. used on a large batch of food). Conditioning is achieved with magnetic fields having the period 24/2¹⁸ hour, ≦+/−1%, and principally in the same way as described in connection with FIG. 3. Said generator can be connected to control means which controls amplitude, period, phase, sequence and ON/OFF.

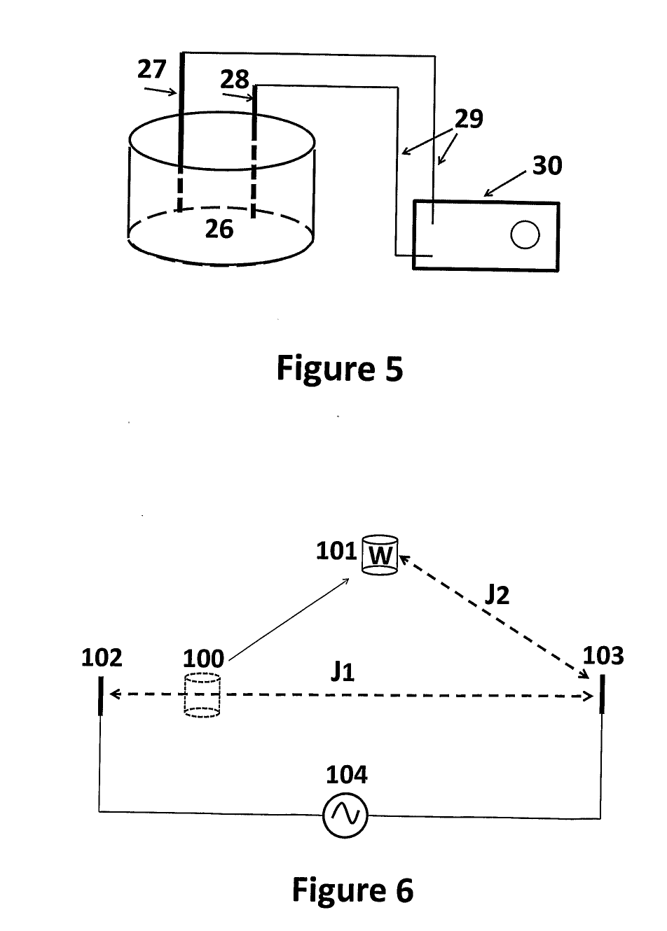

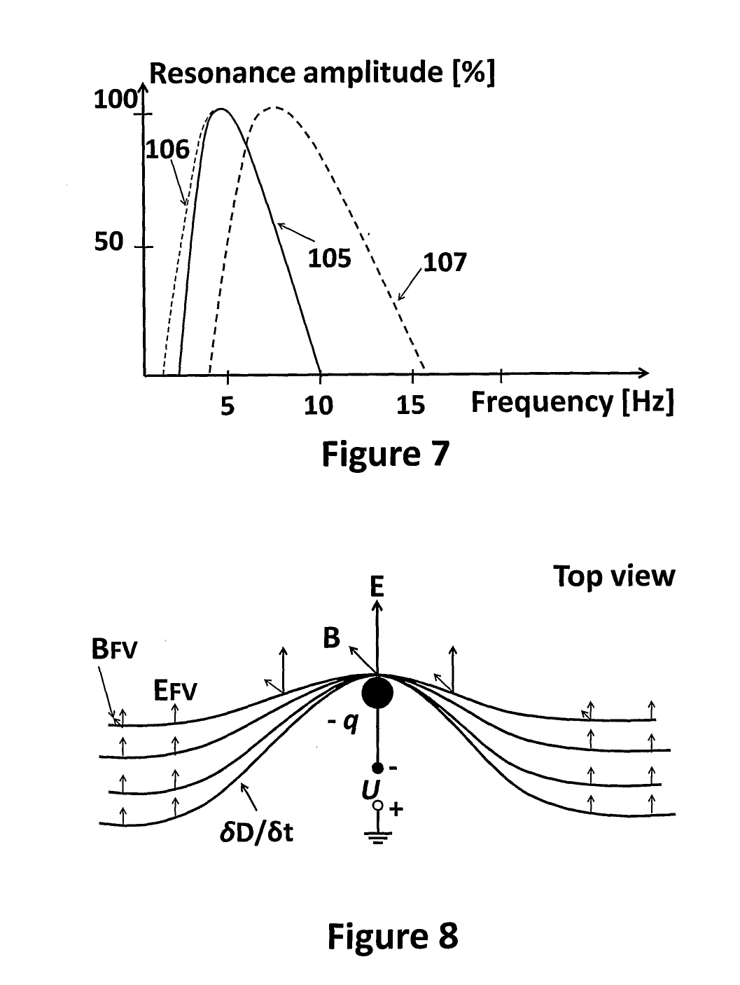

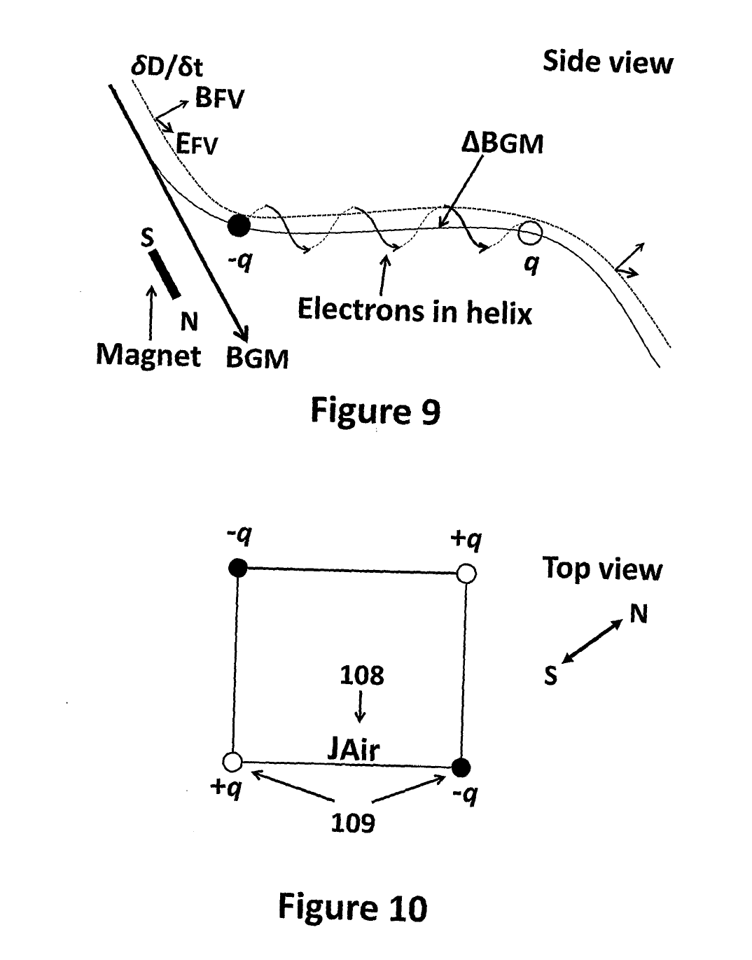

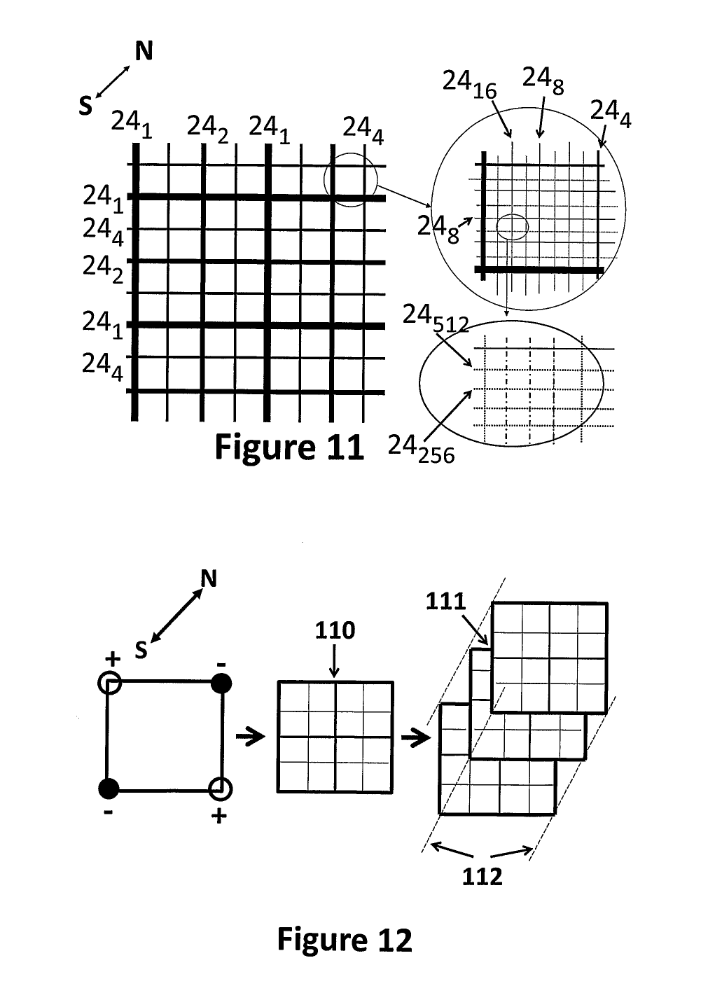

FIG. 5 describes a method by which resonance in water is created by conduction current. Two electrodes (27 & 28) are connected, through wires (29) to a signal generator (30) having the period 24/3·2¹⁷ hour, ≦+/−1%. The electrodes are attached or submerged into the water (26). The method can be used to create resonance in a small part of the human body. In that case two small electrodes are attached (galvanic connection) to the area being treated and the conduction current J is typically 0.1 pA≦J≦10 pA. A large sample of water (ex. a water reservoir) demands larger electrodes or a multiple of parallel electrodes and larger conduction current. Conditioning is achieved with conduction current having the period 24/2¹⁸ hour, ≦+/−1%, and principally in the same way as described in connection with FIG. 3. Said generator can be connected to control means which controls amplitude, period, phase, sequence and ON/OFF.

A method to condition water consists of an electromechanical transducer (ex. a piezoelectric element) which is connected, through wires, to a generator that generates current. The generator frequency f is preferably 0.1 kHz≦f≦5 kHz and the duration time t, 1 s≦t≦10 s. The transducer is attached to or submerged into the water and it generates mechanical waves or sound waves into the water having said frequency and duration. This method is used in combination with the methods described in FIGS. 1, 2, 3, 4 and 5.

1-28. (canceled)

29. A method to create resonance in water at the period 24/(3·2¹⁷) hour, characterized by exposing said water during a limited time to electromagnetic energy having the period 24/(3·2¹⁷) hour, +/−1%, and of a higher amplitude than the amplitude of the corresponding energy in the air, whereby the continued resonance in said water is thereafter maintained by electromagnetic energy in the air having the period 24/(3·2¹⁷) hour.

30. The method according to claim 29, characterized by exposing said water in a first step to electromagnetic energy having the period 24/2¹⁸ hour, +/−1%, and then in a second step exposing said water to electromagnetic energy having the period 24/(3·2¹⁷)hour, +/−1%, alternatively performing the two steps simultaneously, whereby the amplitude of resonance at the period 24/(3·2¹⁷) hour in said water is increased compared to the amplitude of resonance obtained in the method according to claim 29.

31. The method according to claim 29, characterized by that said water includes sea water, lake water, river water, water in wells, water in reservoirs, water in beverages, water in food, water in human body fluids and cells, water in other types of organisms including bacteria, pathogens, viruses and molecule structures consisting of H₂O molecules and ions.

32. A method to limit and/or destroy the ability of organisms, including all types of pathogens, to create resonance with electromagnetic energy having a period deviating from the period 24/(3·2¹⁷) hour, characterized by exposing said organisms to electromagnetic energy according to claim 29 and preferably during 30-60 seconds, which is repeated ≧2 times daily during ≦21 days depending on type of organism, seriousness of the infection and general condition of the human being.

33. The method according to claim 32, characterized in that the organisms include all types of disease causing organisms, all types of pathogens, all types of pathogens causing infections, all types of pathogens causing inflammations, all types of bacteria, all types of viruses, all types of cancer cells and tumors, all types of mould and all types of fungal microbes.

34. The method according to claim 29, characterized in that said electromagnetic energy consist of conduction current or electric fields or magnetic fields or combinations of said energies.

35. The method according to claim 29, characterized by utilizing one or many oscillators, each consisting of a conductor, to create conduction current at one or two periods, wherein a first oscillator, (the period 24/(3·2¹⁷) hour) has conductor length L₃=v·24·60·60/(3·2·2ⁿ) m, 10≦n≦17, and wherein second and third oscillators (the period 24/2¹⁸ hour) have conductor lengths L₁=v·24·60·60/(2·2ⁿ) m, 10≦n≦18, where v is the drift velocity of electrons in said conductor and wherein said oscillator(s) are positioned in a media containing positive ions.

36. The method according to claim 29, characterized in that a magnetic field is used in order to increase, decrease or eliminate conduction current in the air.

37. The method according to claim 29, characterized by exposing said water to a mechanical pulse or mechanical waves, resulting in that the amplitude of resonance in said water at the period 24/(3·2¹⁷)hour is increased compared to the amplitude of resonance obtained in the method according to claim 29.

38. The method according to claim 29, characterized by utilizing one or many oscillators, electromagnetically connected through wires, capacitors or electric fields to a generator having outputs per oscillator, to generate conduction current, and wherein the first oscillator is connected to an output delivering a sinus signal having the period 24/(3·2ⁿ) hour, +/−1%, and the voltage U, 0 V≦U≦100 V RMS, and wherein the second and third oscillators and are connected to outputs delivering sinus signals having the period 24/2ⁿ hour, +/−1%, having 180 degrees phase difference and the voltage U, 0 V≦U≦100 V RMS, and wherein said generator is connected to control means, which enables control of periods, sequence, amplitudes and ON/OFF.

39. A method to limit and/or destroy the ability of organisms, including all types of pathogens, to create resonance with electromagnetic energy in the air having the period 24/(m·2ⁿ) hour, characterized by exposing said organisms continuously or preferably during 24-96 hours to electromagnetic energy of a higher amplitude than the amplitude of the corresponding energy in the air consisting of conduction current, electric fields, magnetic fields or combinations of said energies having the frequency f, 1 Hz≦f≦15 Hz, and excluding at least one of the periods 24/2¹⁸ hour, 24/(3·2¹⁷) hour, 24/(5·2¹⁶) hour, 24/(7·2¹⁶) hour and 24/(9·2¹⁵) hour, +/−5%.

40. The method according to claim 39, characterized in that the organisms include all types of disease causing organisms, all types of pathogens, all types of pathogens causing infections, all types of pathogens causing inflammations, all types of bacteria, all types of viruses, all types of cancer cells and tumors, all types of mould and all types of fungal microbes.

41. Use for the method according to claim 29, to limit and/or destroy organisms including all types of pathogens.

42. The use according to claim 41 wherein the organism includes all types of bacteria, all types of virus, all types of mould, all types of fungal microbes and all types of pathogens.

43. Use for the method according to claim 29, for the treatment of a disease.

44. Use for the method according to claim 29, to cure an illness.

45. The use according to claim 44 wherein the illness is cancer.

46. The use according to claim 44 wherein the illness is caused by any type of pathogen.

47. The use according to claim 44 wherein the illness is caused by pathogens in infections.

48. The use according to claim 44 wherein the illness is caused by pathogens in inflammations.

49. The use according to claim 44 wherein the illness is caused by pathogenic bacteria or bacteria.

50. The use according to claims 44 wherein the illness is caused by viruses.

51. The use according to claims 44 wherein the illness is caused by fungal microbes.

52. The use according to claims 44 wherein the illness is caused by mold.

53. Use for the method according to claim 29 to strengthen the immune system.

54. Use for the method according to claim 29, for preserving food.

55. Use for the method according to claim 29, to improve the quality of water.

56. Use for the method according to claim 29, to influence the molecule structure of water used in industrial processes.
